(19) United States
(12) Patent Application Publication
(10) Pub. No.: US 2018/0221022 A1
(43) Pub. Date: Aug. 9, 2018

(54) RETAINER ASSEMBLY

(71) Applicant: Regents of the University of Minnesota, Minneapolis, MN (US)

(72) Inventors: Adam Choe, Coon Rapids, MN (US); Hannah Bearinger, St. Paul, MN (US)

(21) Appl. No.: 15/891,102

(22) Filed: Feb. 7, 2018

Related U.S. Application Data
(60) Provisional application No. 62/455,907, filed on Feb. 7, 2017.
(60) Provisional application No. 62/474,936, filed on Mar. 22, 2017.
(60) Provisional application No. 62/490,448, filed on Apr. 26, 2017.

(51) Int. Cl. A61B 17/06

(52) U.S. Cl. CPC A61B 17/06166; A61B 2017/0412

(57) ABSTRACT

A fastener includes a first end, an elongate body, and a second end. The first end includes a cylinder having an entry aperture at a terminus, a wall, and an inner lumen aligned with a longitudinal axis of the cylinder. The lumen terminates at the entry aperture. The elongate body is aligned coaxially with the longitudinal axis and contiguous with the cylinder. The second end includes an insertion body aligned coaxially with the elongate body and contiguous with the elongate body. The entry aperture and the lumen are configured to receive the second end. The second end is aligned to penetrate the entry aperture and thereafter enter the lumen.

### CLAIM OF PRIORITY

This patent application claims the benefit of priority of U.S. Provisional Patent Application Ser. No. 62/455,907, filed on Feb. 7, 2017, and claims the benefit of priority of U.S. Provisional Patent Application Ser. No. 62/474,936, filed on Mar. 22, 2017, and claims the benefit of priority of U.S. Provisional Patent Application Ser. No. 62/490,448, filed on Apr. 26, 2017, each of which is hereby incorporated by reference herein in its entirety.

### TECHNICAL FIELD

This document pertains generally, but not by way of limitation, to a knotless fastener such as a knotless suture.

### BACKGROUND

Sutures are used in some surgical procedures to mechanically fasten adjoining structures. By way of examples, a suture can be used to bond an implantable device to tissue or a suture can be used to bond a first tissue to a second tissue.

A traditional suture includes a filamentous material and a manually tied knot. The knot is formed by manipulating the ends of the filament in a particular manner to cinch the suture around the structures to be joined.

Knots can be problematic. For example, a traditional suture does not readily allow for tension adjustment. In addition, a suture has a dimensional thickness that is larger than that of the filament and the increased thickness can be a source of abrasion or irritation. Furthermore knot tying can require a rather high level of skill.

### SUMMARY

An example of the present subject matter includes a retainer device configured as a suture free of a manually formed knot. A device can include a first end having a cylinder configured to receive a second end. The second end is contiguous with the first end and can include a rigid or flexible body. A variety of mechanisms are provided for affixing the device in position. One example includes a friction fit including a male end that is retained circumferentially by a lumen at an opposing end. The inner diameter of the lumen reduces with tension exerted in a longitudinal direction. In one example, barbs or pins between the lumen and the male end engage in a one-way manner to maintain tension.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a view of a device, according to one example.

FIG. 2 illustrates a view of a device, according to one example.

FIGS. 3A and 3B illustrate views of portions of a device, according to one example.

FIG. 4 illustrates a view of portions of a device, according to one example.

FIG. 5A illustrates a view of a portion of a device, according to one example.

FIG. 5B illustrates a view of a portion of a device, according to one example.

FIG. 6 illustrates a view of a portion of a device, according to one example.

FIG. 7 illustrates a view of a portion of a device, according to one example.

FIG. 8 illustrates a view of portions of a device, according to one example.

FIG. 9 illustrates a view of a device in tissue, according to one example.

FIG. 10 illustrates a flowchart of a method, according to one example.

### DETAILED DESCRIPTION

FIG. 1 illustrates a view of device 70A according to one example. Device 70A can include a fastener configured as flexible filament or as a rigid member that has ends that are coupled, either to itself or to another fastener, in a manner that does not require formation of a knot.

In the figure, device 70A includes first end 100A and second end 200A spaced apart by elongate body 300A.

First end 100A includes cylinder 120A having an entry aperture 130A disposed at terminus 140A of device 70A. Cylinder 120A includes cylinder wall 150A. Cylinder wall 150A defines inner lumen 160A. Cylinder 120A has a longitudinal axis 170 aligned on the center of the lumen diameter.

Elongate body 300A is aligned coaxially with longitudinal axis 170 and is coupled to first end 100A. In one example, elongate body 300A is contiguous with first end 100A.

Second end 200A includes insertion body 208 aligned coaxially with longitudinal axis 170 and is coupled to elongate body 300A. In one example, second end 200A is contiguous with elongate body 300A or is contiguous with first end 100A. Entry aperture 130A and lumen 160A are configured to receive the second end 200A. Second end 200A can be configured or manipulated to penetrate the entry aperture 130A and thereafter enter inner lumen 160A.

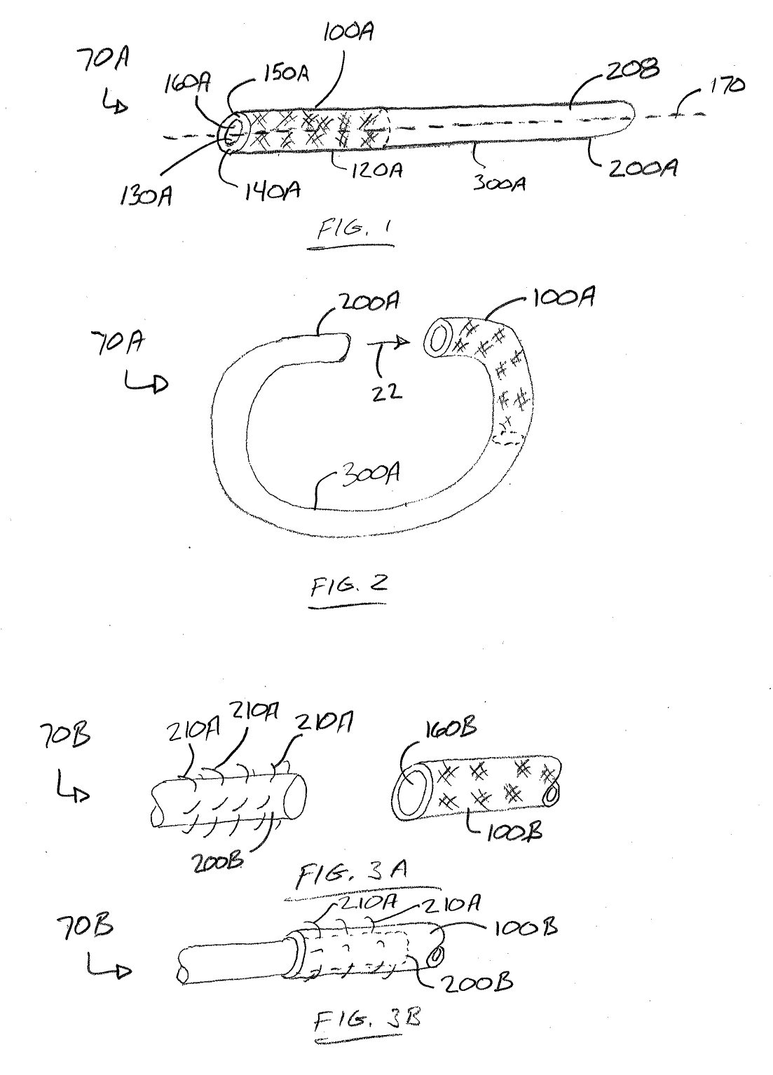

FIG. 2 illustrates a view of device 70A, according to one example. In the figure, second end 200A is disposed for entry into first end 100A. Movement of second end 200A in the direction indicated by arrow 22 will bring second end 200A into the inner lumen of first end 100A. In the figure, elongate body 300A can be disposed to encircle a joint between adjacent tissue segments or between other structure.

FIGS. 3A and 3B illustrate views of portions of a device, according to one example. In the figures, first end 100B and second end 200B of device 70B are illustrated. As shown in FIG. 3A, second end 200B includes a plurality of barbs, some of which are denoted as barb 210A. Barb 210A can include a wire segment affixed in manner to radial project from second end 200B. Barb 210A can have an angular configuration relative to axis 170 in a manner to engage with first end 100B. Second end 200B is configured to enter lumen 160B in a one-way manner such that the force for entry into the lumen is substantially less than the force required to separate first end 100B and second end 200B. Upon entering lumen 160B, barb 210A readily deflects in a manner to enable sliding with little resistance. Upon separating or disengagement, barb 210A can engage with a wall of inner lumen 160B and, as shown in FIG. 3B, barb 210A extends into and passes through the wall of lumen 160B.

Barbs 210A can have a curvature or have an initial alignment that lays substantially parallel with a longitudinal axis. When under tension, barbs 210A can be arranged in a radial spoke manner. Barbs 210A can be fabricated of metal or polymer and can be manufactured, for example, using an extrusion and cutting process or a molding operation.

In one example, barbs 210A are configured to have nap. Nap refers to the alignment of fibers in which movement in one direction encounters more fiber ends and movement in another direction encounters more fiber sides.

FIG. 4 illustrates a view of portions of device 70C, according to one example. In the figure, first end 100C and second end 200C of device 70C are illustrated in an engaged position. In the figure, second end 200C is positioned within lumen 160C. In this example, second end 200C can be fused or bonded in a manner to solidify a portion as denoted by longitudinal markings 206.

Disengagement is associated with movement of second end 200C in a direction denoted by arrow 23 while first end 100C remains substantially stationary, with movement of first end 100C in a direction denoted by arrow 24 while second end 200C remains substantially stationary, or by movement of both first end 100C and second end 200C in the manner denoted by the corresponding arrows. Engagement is associated with corresponding movement in directions opposite to that shown by arrow 23 and arrow 24.

In one example, movement in the direction of disengagement causes reduction in a diameter of lumen 160C, thereby increasing friction between first end 100C and second end 200C. Similarly, movement in the direction of engagement tends to cause an increase in a diameter of lumen 160C, thereby reducing friction.

FIG. 5A illustrates a view of second end 200D of a portion of a device, according to one example. In the figure, second end 200D includes a plurality of formed barbs 212. Second end 200D and barbs 212 can be formed of a polymer or other material. Barbs 212 can be localized or distributed about the length of second end 200D as well as along a length of elongate body 300A. Barbs 212 are configured to engage in one-way manner with a corresponding lumen of a first end, such as first end 100A.

FIG. 5B illustrates a view of second end 200E of a portion of a device, according to one example. In the figure, second end 200E includes bulbous end 220. Second end 200E and bulbous end 220 can be formed of a polymer or other material. Bulbous end 220 can be configured to engage in one-way manner with a corresponding lumen of a first end, such as first end 100A.

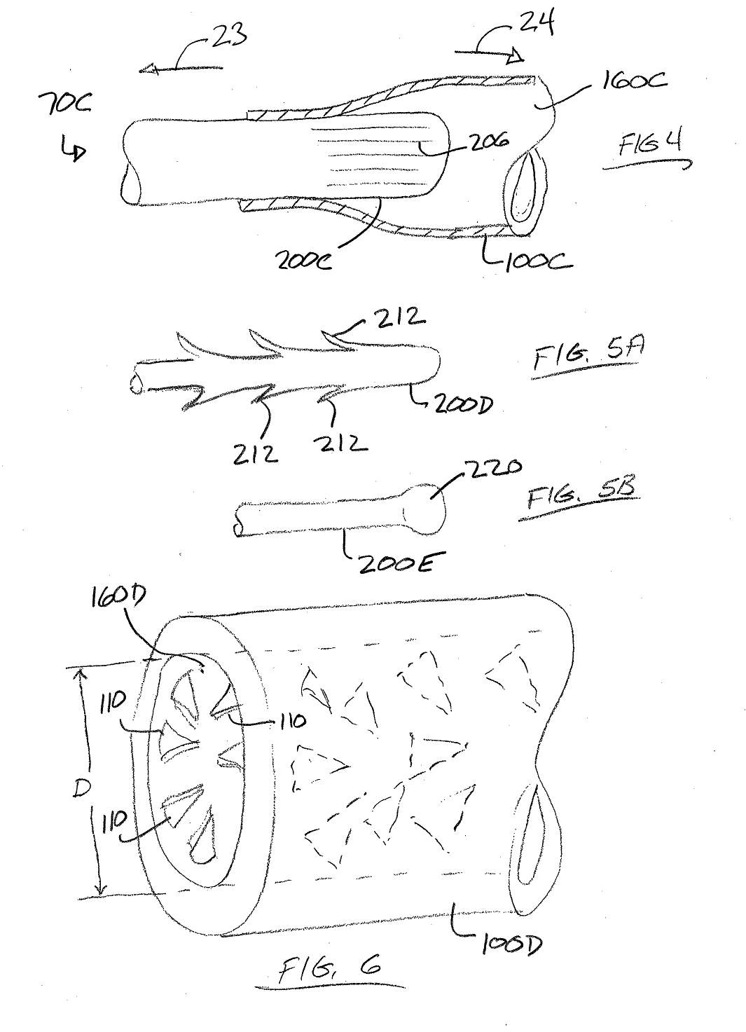

FIG. 6 illustrates a view of first end 100D of a portion of a device, according to one example. In the figure, diameter D of lumen 160D is indicated. As noted elsewhere in this document, and in one example, a compressive force exerted on first end 100D in a direction along the longitudinal axis (such as axis 170) tends to increase diameter D and a tension force exerted on first end 100D in a direction along the longitudinal axis (such as axis 170) tends to decrease diameter D. In one example, first end 100D includes a woven fabric having a plurality of filaments aligned on a bias. In one example, such as that shown, first end 100D includes a plurality of engagement features 110 disposed on an interior wall surface of lumen 160D. Engagement feature 110, in the example shown, includes a polymer having a triangular configuration in which an apex of a triangle engages with a second end in a one-way manner.

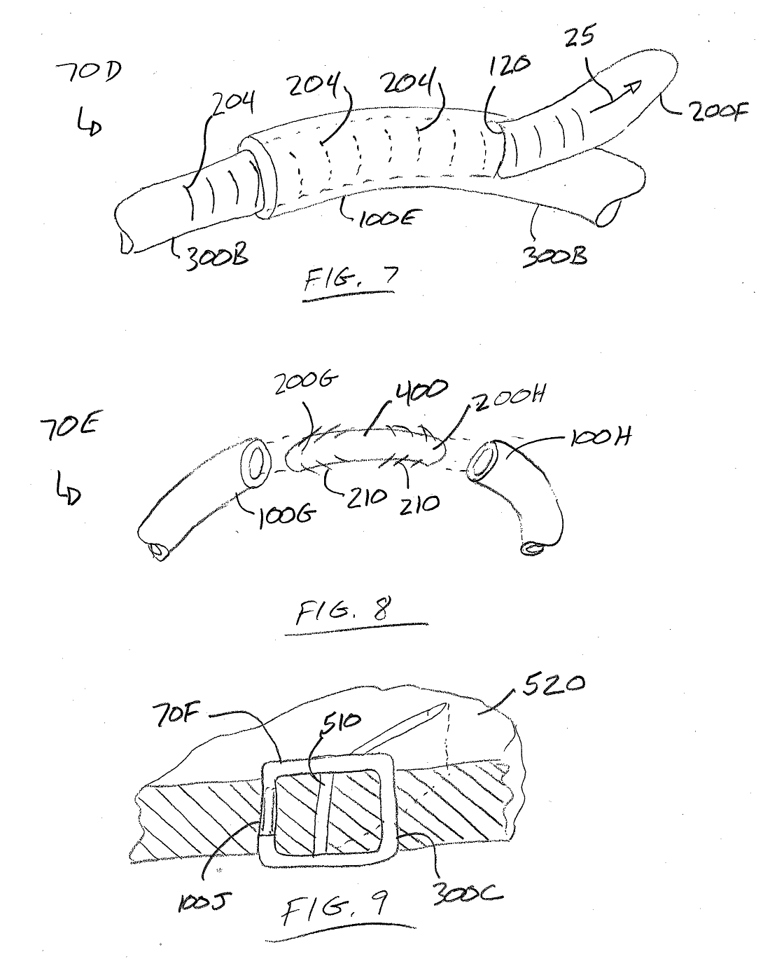

FIG. 7 illustrates a view of a portion of device 70D, according to one example. In the figure, device 70D includes second end 200F engaged with first end 100E. Elongate body 300B is coupled to first end 100E and coupled to second end 200F. In the example illustrated, engagement entails passing second end 200F into first end 100E by relative movement in the direction shown by arrow 25. Second end 200F passes through side aperture 120 of first end 100E. Side aperture 120 is in communication with a lumen of first end 100E.

In one example, a friction fit associated with a bias woven fabric retains suitable tension on device 70D and ensures retention of second end 200F relative to first end 100E.

In one example, features 204 disposed along a length of second end 200F (and in one example, along a portion of a length of elongate body 300B) are configured to engage a corresponding feature of first end 100E in a one-way manner. For example, a corresponding feature can include a tang or spring element to engage a ridge of feature 204.

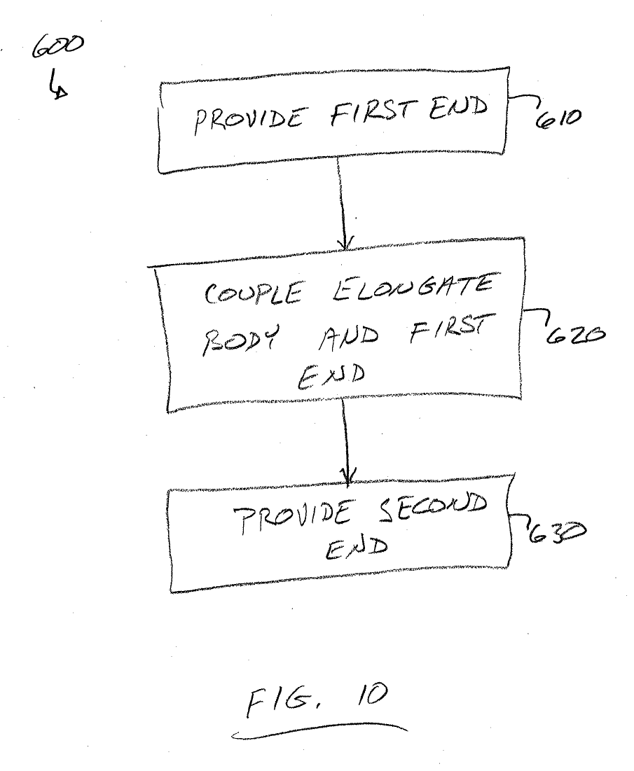

FIG. 8 illustrates a view of portions of device 70E, according to one example. Device 70E includes first end 100G and first end 100H, each of which includes a corresponding lumen. Device 70E includes connector 400 having second end 200G and second end 200H disposed at opposite ends. Connector 400, in the example shown, includes a plurality of barbs 210B disposed along a length. Barbs 210B and second ends 200G and 200H engage with corresponding aspects of first ends 100G and 100H in a one-way manner.

FIG. 9 illustrates a view of device 70F disposed in tissue 520, according to one example. Device 70F includes a first end 100J and elongate body 300C and in the figure, device 70F binds joint 510. Device 70F can be rigid or flexible to allow manipulation and placement as shown.

FIG. 10 illustrates a flowchart of method 600, according to one example. In the example illustrated, at 610, method 600 includes providing a first end including a cylinder having an entry aperture at a terminus, a wall, and an inner lumen aligned with a longitudinal axis of the cylinder, the lumen terminating at the entry aperture. At 620, method 600 includes coupling an elongate body to the first end. In one example, coupling includes aligning coaxially with the longitudinal axis and contiguous with the cylinder. At 620, method 600 includes providing a second end including an insertion body aligned coaxially with the elongate body and contiguous with the elongate body. The entry aperture and the lumen are configured to receive the second end. The second end is aligned to penetrate the entry aperture and thereafter enter the lumen.

Method 600 can also include variations. For example, at 610, providing a first end can include forming the cylinder of a flexible woven fabric. As another example, at 620, coupling the elongate body to the first end can include forming the elongate body and the first end using a segment of elongate elastic material. In addition, at 630, providing the second end can include fusing a portion of an elongate elastic material.

### Various Notes

In one example, the elongate body is substantially omitted in that the first end and the second end are co-terminus.

According to one example, a user-selectable insertion depth of a second end relative to a first end enables selection of tension on the suture.

One example includes a first end having a cylinder wall of woven fabric. The woven fabric can be configured on a bias in which compression tends to increase a diameter of the cylinder and a tension tends to decrease the diameter of the cylinder.

One example includes a first end having a cylinder wall of non-woven fabric. In one example, the first end is substantially rigid and the corresponding second end includes a male end having solid or hollow structure with anchors or barbs distributed about the periphery. The anchors or barbs are configured to deploy and to engage with the walls of the female end.

Engagement of the first end and the second end of an example device can cause a decrease in the diameter of the inner lumen. The second end is subjected to passive radial tension with reduction in the diameter of the inner lumen. In one example, the anchors or barbs disposed on the second end extend out through the wall of the cylinder of the first end.

In one example, a device is wholly or partially fabricated of a biocompatible material or a bioabsorbable material. For example, a device can include a synthetic material such as polyethylene.

One example can include a plurality of barbs distributed along approximately a third of the overall length and a hollow cylinder over two thirds of the overall length. Other arrangements are also contemplated, such as half and half.

In one example, a device is fabricated of a monofilament absorbable material. An example can include a synthetic material such as polyglyconate, a copolymer of glycolic acid and trimethylene carbonate. In one example, a device includes braided nylon having a diameter of 1.18 mm.

In one example, a plurality of barbs are distributed on an inner wall of a lumen of a first end. In one example, a plurality of barbs are distributed on an outer surface of a second end. The barbs can be configured to pass through a wall of a woven fabric wall or can be configured to engage without passing through the thickness of the wall.

In one example, the second end includes a male end fabricated by fusing multiple filaments.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A fastener comprising:
a first end including a cylinder having an entry aperture at a terminus, a wall, and an inner lumen aligned with a longitudinal axis of the cylinder, the lumen terminating at the entry aperture;
an elongate body aligned coaxially with the longitudinal axis and contiguous with the cylinder; and
a second end including an insertion body aligned coaxially with the elongate body and contiguous with the elongate body, the entry aperture and the lumen configured to receive the second end and wherein the second end is aligned to penetrate the entry aperture and thereafter enter the lumen.

2. The fastener of claim 1 wherein the cylinder wall is flexible.

3. The fastener of claim 1 wherein the cylinder wall is fabric.

4. The fastener of claim 1 wherein the cylinder wall includes woven fabric.

5. The fastener of claim 1 wherein the cylinder wall includes fabric in a helically wound braid.

6. The fastener of claim 1 wherein a compressive force exerted on the cylinder and aligned with the longitudinal axis is associated with an increase in a diameter of the inner lumen.

7. The fastener of claim 1 wherein the second end is frictionally retained in the lumen.

8. The fastener of claim 1 wherein the insertion body include a plurality of barbs aligned to retain the insertion body in the lumen.

9. The fastener of claim 1 wherein a barb of the plurality of barbs includes at least one of metal or a polymer.

10. The fastener of claim 1 wherein the insertion body includes a bulbous end.

11. The fastener of claim 1 further including a plurality of barbs disposed on an inner surface of the cylinder wall, the barbs aligned to retain the insertion body in the lumen.

12. The fastener of claim 1 wherein the cylinder is substantially rigid.

13. The fastener of claim 1 wherein the elongate body is rigid.

14. The fastener of claim 1 further including a side aperture in communication with the inner lumen, the side aperture disposed along the longitudinal axis.

15. A method of forming a fastener comprising:
providing a first end including a cylinder having an entry aperture at a terminus, a wall, and an inner lumen aligned with a longitudinal axis of the cylinder, the lumen terminating at the entry aperture;
coupling an elongate body to the first end in a manner aligned coaxially with the longitudinal axis and contiguous with the cylinder; and
providing a second end including an insertion body aligned coaxially with the elongate body and contiguous with the elongate body, the entry aperture and the lumen configured to receive the second end and wherein the second end is aligned to penetrate the entry aperture and thereafter enter the lumen.

16. The method of claim 15 wherein providing a first end includes forming the cylinder of a flexible woven fabric.

17. The method of claim 15 wherein coupling the elongate body to the first end includes forming the elongate body and the first end using a segment of elongate elastic material.

18. The method of claim 15 wherein providing the second end includes fusing a portion of an elongate elastic material.
